(19) United States
(12) Patent Application Publication
(10) Pub. No.: US 2023/0054550 A1
(43) Pub. Date: Feb. 23, 2023

(54) COMPOSITION AND METHOD FOR CATALYTIC REDUCTION OF CARBON DIOXIDE OR CARBOHYDRATE

(71) Applicant: JIAXING UNIVERSITY, JIAXING, ZHEJIANG PROVINCE (CN)

(72) Inventors: XUEBO CAO, JIAXING, ZHEJIANG PROVINCE (CN); KAI WANG, JIAXING, ZHEJIANG PROVINCE (CN); HANZHANG CAO, JIAXING, ZHEJIANG PROVINCE (CN)

(21) Appl. No.: 17/800,188

(22) PCT Filed: Jun. 8, 2020
(86) PCT No.: PCT/CN2020/094810

§ 371 Date: Aug. 16, 2022

(30) Foreign Application Priority Data
Feb. 20, 2020 (CN) 202010103825.1

(51) Int. Cl. B01J 31/02; C07C 1/22; C01B 32/40; B01J 35/00; B01J 23/06; B01J 23/72; C07C 1/12; B01J 23/755; B01J 23/50; B01J 23/75; B01J 23/14; B01J 21/02

(52) U.S. Cl. CPC B01J 31/0244; B01J 21/02; B01J 23/06; B01J 23/14; B01J 23/50; B01J 23/72; B01J 23/75; B01J 23/755; B01J 35/0006; C01B 32/40; C07C 1/12; C07C 1/22; B01J 2231/64; B01J 2231/625

(57) ABSTRACT

Embodiments of the present invention relates to integrated catalyst systems and associated processes that directly converts carbon dioxide or carbohydrate to CO, methane, or other valuable chemicals at room temperature and atmospheric pressure, requiring no extra energy. The integrated catalyst systems are comprised of nitrogenous heterocyclic compounds and at least two metal elements, wherein one metal element needs to be active than the other one. The integrated catalyst systems can be applied to reduce carbon dioxide and carbohydrate at room temperature with considerable conversion efficiency. The reduction process involves the steps of: a) nitrogenous heterocyclic compounds performance as solvent/major catalyst, dual component as reducing agent / co-catalyst; b) introducing the above integrated catalysts into the reactor full of CO₂ or carbohydrate, and keeping stirring the reacting system for 1 to 4 hours, without any illumination or heating; c) CO, methane, or other reduction product is achieved with a conversion efficiency of about 100%; d) the reduction products are gases, which can be directly separated from the system without any additional separation process or involving additional chemicals.

This is a U.S. national stage application of PCT Application No. PCT/CN2020/094810 under 35 U.S.C. 371, filed Jun. 8, 2020 in Chinese, claiming priority of Chinese Application No. 202010103825.1, filed Feb. 20, 2020, all of which are hereby incorporated by reference.

### FIELD OF THE INVENTION

The present invention relates to chemical catalytic technology, wherein, integrated catalysts and processes for catalytic reduction of carbon dioxide and carbohydrate.

### BACKGROUND OF THE INVENTION

More and more attention has been paid to the emission of CO₂, since the global warming and environment pollution became considerably imminent and serious. (Friedlingstein, P.; Houghton, R. A.; Marland, G.; Hackler, J.; Boden, T. A.; Conway, T. J.; Canadell, J. G.; Raupach, M. R.; Ciais, P.; Quere, C. L. Nature Geosci. 2010, 3, 811). As the primary greenhouse gas, the content of CO₂ will decide the tendency of global climate (Schrag, D. P. Science 2007, 315, 812). At present, the total energy consumed by human beings is 14TW/year, and the total energy consumption will be three times of the present data in 2050 (Biernat K, Malinowski A, Gnat M. INTECH; 2012, 123), wherein, fossil fuel will be up to 83% of the total energy, and the consumption of fossil fuel directly resulted in more CO₂ emission. On average, scientists use physical or chemical means to capture thousands of tons of carbon dioxide every year, but they still cannot effectively solve the problem (Schrag, D. P. Science 2007, 315, 812.; Chen, B.; Nishio, M.; Song, Y. C.; Akai, M. Energy Procedia 2009, 1, 4969). The resource utilization of CO₂ comprises physical utilization, biological utilization, and chemical utilization, wherein, physical utilization, as a recycling process, is capable of curbing carbon emission (Quadrelli E A, Centi G, Duplan J L, et al. ChemSusChem,2011,4(9) ,1194); Biological utilization can be applied to convert CO₂ to bio-fuel and fertilizer through absorption and fixation of CO₂ by green plants or microbe (Costentin C, Robert M, Saveant J M. Chemical Society Reviews, 2013, 42 ( 6), 2423); Chemical utilization, as the most efficient technology, can be employed to convert CO₂ to premium chemical products through chemical reactions (Markewitz P, Kuckshinrichs W, Leitner W, et al. Energy & Environmental Science, 2012, 5(6),7281). However, the thermodynamic stability and kinetic inertness of carbon dioxide make its efficient conversion and utilization difficult. Therefore, the research and development of carbon dioxide conversion and utilization technology has become the key to improving the utilization of carbon dioxide resources.

At present, chemistry reduction for the conversion and utilization of CO₂ mainly falls into four groups: thermochemistry reduction, biochemistry reduction, photochemistry reduction, electrochemistry reduction, wherein, the thermochemical reduction method is mainly a catalytic hydrogenation reaction in practical industrial applications, and there are problems such as excessive reaction temperature, inability to directly separate some by-products, and catalyst activity and stability need to be improved. Photochemical reduction and photocatalytic reduction, as energy-saving, pollution-free and mild reaction conditions, have been favored by researchers in recent years, but there are still problems such as low solar energy utilization and low conversion efficiency. Electrocatalysis also suffers from problems such as high power consumption, low catalytic efficiency, slow conversion rate, and poor selectivity.

For example, the invention patent application with publication number CN105080564A discloses a catalyst for converting carbon dioxide to carbon monoxide, which includes the following components in weight percentage: 2% to 30% of Mn oxide, 0.5% to 10% of Ce or La At least one oxide, 0.5% to 5% of Cu oxide, 1% to 5% of alkali metal and 50% to 96% of composite carrier, wherein the composite carrier includes 5% to 39% by weight ZnO and 61% to 95% Al₂O₃. However, in this application, carbon dioxide and hydrogen are used as the raw material gas, and the raw material gas contacts the catalyst at a reaction temperature of 400 to 580° C., a reaction pressure of 1 to 3 Mpa, and a volume ratio of H₂/CO₂ (1.2 to 3): 1. The reaction yields carbon monoxide.

For another example, the invention patent application with publication number CN109731578A discloses a carbon dioxide hydrogenation conversion catalyst and method. The catalyst is CuIn@SiO₂ with a core-shell structure, with CuIn alloy as the core and porous SiO2 as the shell, and the CuIn alloy is coated on In the porous SiO2 shell, the mass fraction of the porous SiO2 in the catalyst is 50-80 wt %. A preparation method of a carbon dioxide hydroconversion catalyst, the carbon dioxide hydroconversion catalyst CuIn@SiO₂ uses polyvinylpyrrolidone (PVP) as a coating agent and cetyltrimethylammonium bromide (CTAB) as a structure directing agent, obtained by two solvothermal treatments and reduction in a hydrogen atmosphere.

### SUMMARY OF THE INVENTION

With regard to the deficiency existing in the prior art, the present invention relates to an integrated catalytic composition and processes for catalytic reduction of carbon dioxide and carbohydrate, which enjoy several advantages over that in the prior art, wherein, with the improved catalytic composition, CO₂ can be reduced to CO and methane at room temperature and in mild reaction conditions, also with high conversion efficiency.

A composition for catalytic reduction of carbon dioxide or carbohydrate, comprises nitrogen heterocyclic compounds and at least two metal elements, wherein, one metal element, performing as reactant, enjoys much more reactivity than the other metal element which acts as co-catalyst.

Preferably, the nitrogen heterocyclic compounds comprise at least one of the following reagents: imidazole, 1-methylimidazole, 1-ethylimidazole, 1-ethyul-3-methylimidazolium tetrafluroborate salt, 4-methylimidazole, 1-allyimidazole, 2-methylimidazole, 1-butyl-3-methyl imidazolium bromide salt, 1-benzylimidazole, histamine, 1-butylimidazole, acetonitrile(1-imidazole group), 1,2-dimethylimidazole, 1-acetylimidazole, and 1,2,4-triazole, wherein all nitrogen heterocyclic compounds except 1,2,4-triazole are imidazoles with imidazole ring. The ratio between the nitrogen-containing heterocyclic compound and the metal element as the reactant can be used according to the corresponding ratio in the chemical reaction formula. Of course, adding more of one of them does not affect the progress of the reaction, and the effect is similar.

Preferably, the metals in the catalytic composition comprise two different metal elements. Generally, the more the kinds of metal elements employed, the better the catalytic efficiency, while the cost will increase correspondingly. CO₂ cannot be reduced and converted to CO or methane since only one type of metal elements was employed, wherein, they are Copper, Silver, Tin, Nickel, and Lead, which are poor in chemical activity. On the other hand, CO₂ can be reduced and converted to CO or methane if one kind of metal elements was used and they are Zinc, Ferrous, Aluminum, and Magnesium, which are chemical active. However, the conversion efficiency for this single active metal element catalytic system is less than 20% of that achieved in double metal elements catalytic system. Therefore, the system of double metal elements performs the key part in the integrated catalytic reduction system for conversion of CO₂ and carbohydrate.

Preferably, as co-catalyst, the chemical inert metal elements family (M₁) comprises Tin, Copper, Silver, Nickel, Cadmium, Cobalt and Lead, wherein, only with slight amount, they can be applied repeatedly without any replenishment. As reactant, the active metal elements group (M₂) includes Zinc, Iron, Aluminum, Manganese, Magnesium, Nickel and Tin, wherein, they are consumed in the reaction. The preferred ratio for M₁ to M₂ is 1: 0.25 - 250, wherein, the conversion of CO₂ efficiency is desirable.

The degree of activity of metals is relative, a metal can be the one with higher degree of activity in one combination, and can also be the side with lower degree of activity in another combination. The degree of metal activity reflects the standard electrode potential of the metal. The standard electrode potential of the metal is as follows:
Magnesium, E(Mg/Mg²⁺) = -2.372V,
Aluminium, E(Al/Al³⁺) = -1.662V,
Zinc, E(Zn/Zn²⁺) = -0.76 V,
Iron, E(Fe/Fe²⁺) = -0.447V,
Nickel, E(Ni/Ni²⁺) = -0.257V,
Manganese, E(Mn/Mn²⁺) = -1.185V,
Tin, E(Sn/Sn²⁺) = -0.138V,
Silver, E(Ag/Ag⁺) = 0.7996V,
Copper, E(Cu/Cu²⁺) = 0.337V.
Cadmium, E(Cd/Cd²⁺) = -0.352V,
Cobalt, E(Co/Co²⁺) = -0.28 V,
Lead, E(Pb/Pb²⁺) = -0.128 V,

The present invention relates to the application of the integrated composition in the catalytic reduction of CO₂ and carbohydrate.

The present invention also provides a method for catalytic reduction of carbon dioxide or carbohydrates, comprising the steps of: mixing a substrate with the composition and reacting to produce carbon monoxide and/or methane.

Preferably, when the nitrogen-containing heterocyclic compound is solid at normal temperature, the nitrogen-containing heterocyclic compound is dissolved in a solvent. The reaction of the present application can be realized at room temperature, but for some nitrogen-containing heterocyclic compounds that are solid at room temperature, it is necessary to use a solvent to dissolve, so as to facilitate sufficient contact with the reaction substrate. However, the solvent itself only plays the role of dissolving and does not participate in the entire reaction process, so any solvent that can dissolve the corresponding nitrogen-containing heterocyclic compound can be used. More preferably, the solvent is water, methanol or ethanol.

Preferably, as the reaction substrate, CO₂ can be from pure carbon dioxide or from waste gas full of CO₂.

The present invention described the improved catalytic composition by which CO₂ and carbohydrate can be reduced at room temperature, wherein, the reaction conditions were moderate and the conversion efficiency was high. The catalytic reduction process comprises the following steps: a) nitrogenous heterocyclic compounds (imidazole, 1-methylimidazole, 1-ethylimidazole, 1-ethyul-3-methylimidazolium tetrafluroborate salt, 4-methylimidazole, 1-allyimidazole, 2-methylimidazole, et al.) performed as solvent/major catalyst, dual component of metal elements as reducing agent / co-catalyst; b) introducing the above integrated catalysts into the reactor full of mixture of CO₂ and carbohydrate, keeping stirring the reacting system for 1 to 4 hours, without any illumination or heating; c) CO, methane, and other reduction products are achieved with a conversion efficiency of about 100%; d) these reduction products are gases, which can be segregated without any solvents.

### BRIEF DESCRIPTION OF THE DRAWINGS

FIG. 1 illustrates the gas chromatogram of the cylinder gas before the reaction in embodiment 1, wherein, (a) is the gas chromatogram of the standard gas consisting of 2000ppm carbon monoxide, 2000ppm methane, and 2000ppm carbon dioxide; (b) gas chromatogram of the gas in the bottle before reaction.

FIG. 2 shows the detection results of the gas in the bottle after the reaction in Embodiment 1, wherein (a) is the gas chromatogram of the gas in the bottle after the reaction, and (b) is the combustion diagram of the gas in the bottle.

FIG. 3 shows the free radical signal diagram of catalyst M₁ and ImZ in embodiment 1, wherein (a) is the free radical signal diagram after M₁ reacts with ImZ, and (b) is the free radical signal diagram after M₁ reacts with ImZ and carbon dioxide is introduced.

FIG. 4 shows the XRD results of the reaction products in embodiment 1.

FIG. 5 shows the crystal structure of the reaction product in embodiment 1.

FIG. 6 illustrates the gas detection results in the bottle after reaction in Embodiment 2, wherein, (a) is the gas chromatogram after reaction with ionic liquid, and (b) is the combustion diagram of the gas in the bottle.

FIG. 7 summarizes a schematic diagram of carbon dioxide reduction by imidazole + bimetal system

### DETAILED DESCRIPTION OF THE PREFERRED EMBODIMENTS

Reactor: In the experiment, an air-tight reaction flask with a volume of one liter was used as the reactor for the catalytic reduction reaction.

### Embodiment 1: Reducing Carbon Dioxide to Produce Carbon Monoxide

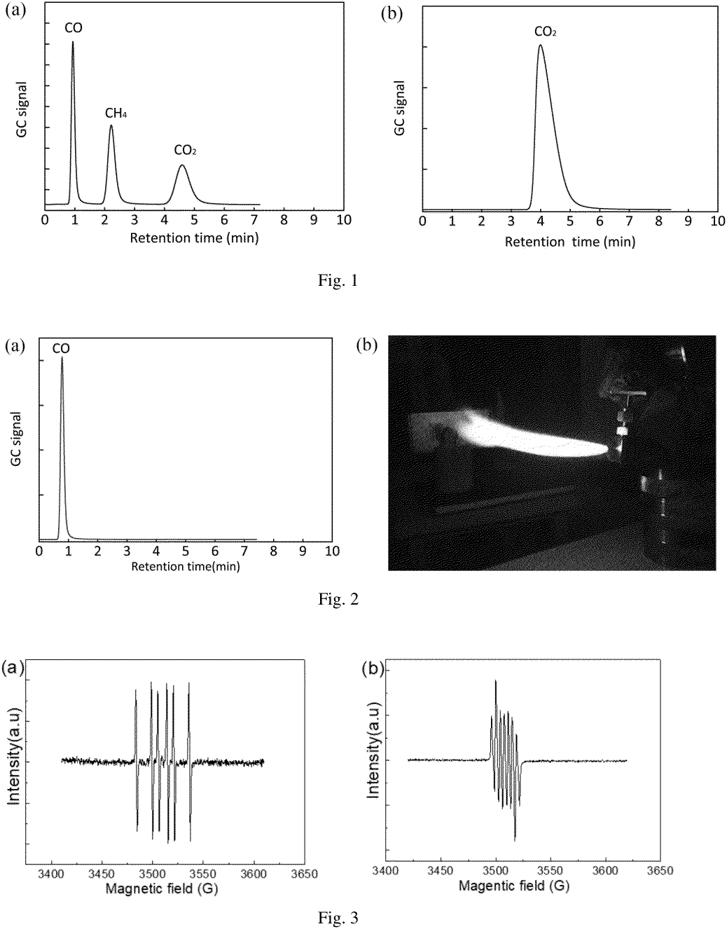

At room temperature, 60 milliliter of 1-methylimidazole, 2.5 gram of zinc powder and 0.5 gram of copper powder were put into a one liter reaction flask. The air in the flask was removed completely by vacuumizing, keep introducing ultra pure CO₂ (99.999%) into the flask until the pressure in the flask approached 0.1 - 0.3 MPa, then the flask was closed. The gas chromatographic instrument was applied to analyze the composition in the flask, the chromatographic peak appeared around t = 4.5 min (FIG. 1b), the gas was identified as carbon dioxide by comparison with the standard gas (FIG. 1a). Then the switch of mini fountain pump was turned on, wherein the imidazole suspension with metallic power was scattered in the flask and thoroughly mixed with CO₂, and series chemical reactions took place. After continuous reaction for 2 hours, the gas in the flask was extracted for GC analysis. The chromatographic peak was found appearing near t=0.9 min (FIG. 2a), while the chromatographic peak near t=4.5 min (the characteristic peak for CO₂) disappeared completely, suggesting that CO₂ was converted to carbon monoxide. Moreover, the carbon monoxide gas burned violently (FIG. 2b) and emitted a blue flame characteristic of carbon monoxide. Combustion experiments further confirmed that the innovative system can efficiently and completely reduce carbon dioxide, and the converted carbon monoxide can be applied as fuel.

FIG. 3 (a) shows the free radical signal obtained by analyzing EPR of the stirred solution of 1-methylimidazole and copper powder with DMPO trapping agent; (b) the free radical signal obtained by analyzing EPR of the stirred mixture of 1-methylimidazole and copper powder with carbon dioxide injected into the solution and DMPO trapping agent added to solution. (Beijing E Testing Company is commissioned to conduct analysis)

Table 1

Organic Element Analysis (C, N, H)

| sample | weight (mg) | method | C [%] | N [%] | H [%] |
|---|---|---|---|---|---|
| Guide sample: Sulfanilamide | 3.0480 | 2 mg 80 s | 41.81 | 16.25 | 4.650 |
| Test #1 | 2.6300 | 2 mg 80 s | 34.13 | 17.38 | 4.117 |
| Test #2 | 2.8200 | 2 mg 80 s | 34.17 | 17.33 | 4.100 |

Table 2

Element Oxygen (O) Analysis

| sample | weight (mg) | method | O [%] |
|---|---|---|---|
| Guide sample: Benzoic acid) | 2.2330 | CO-IRMS | 26.200 |
| Test #1 | 3.4220 | CO-IRMS | 25.106 |
| Test #2 | 3.6380 | CO-IRMS | 25.165 |

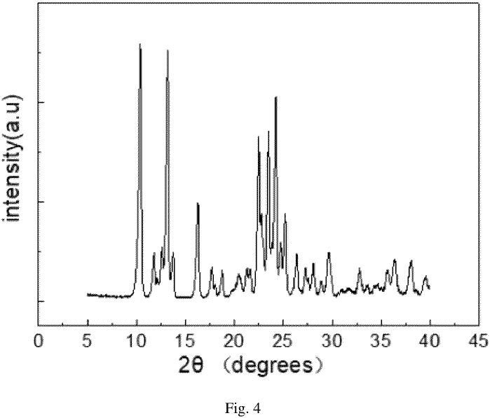

FIG. 4 illustrates the XRD diffraction patterns of the product (white powder) of the reaction, and table 1 and table 2 (test #1 and test #2 are the repeated test for the same sample) (Beijing E Testing Company is commissioned to conduct analysis).

### Embodiment 2: Reducing Carbon Dioxide to Produce Methane

At room temperature, the ionic liquid (60 milliliter of 1-ethyul-3-methylimidazolium tetrafluroborate salt, 2.5 gram of zinc powder and 0.5 gram of copper powder) was put into a one liter reaction flask. The air in the flask was removed completely by vacuumizing, keep introducing ultra pure CO₂ (99.999%) into the flask until the pressure in the flask approached 0.1 - 0.3MPa, then the flask was closed. When the gas in the bottle was confirmed to be pure carbon dioxide analyzed by gas chromatography, a mini fountain pump was switched on, wherein the ionic liquid was scattered in the flask and thoroughly mixed with CO₂, and series chemical reactions took place. After continuous reaction for 3 hours, the gas in the flask was extracted for GC analysis. The chromatographic peak was found appearing near t=2.2 min (FIG. 6a), while the chromatographic peak near t=4.5 min (the characteristic peak for CO₂) disappeared completely, suggesting that CO₂ is converted to methane. Moreover, the methane gas burned violently (FIG. 6b) and emitted a blue flame characteristic of methane. Combustion experiments further confirmed that the innovative liquid system (1-ethyul-3-methyliminazolium tetrafluroborate salt + copper + zinc) can efficiently and completely reduce carbon dioxide, and the converted methane can be applied as fuel.

### Embodiment 3: Reduction of Carbon Dioxide to Produce a Mixture of Carbon Monoxide and Methane

At room temperature, 60 milliliter of methanol, 8 gram of imidazole, 2.5 gram of zinc powder and 0.1 gram of silver powder were put into a one liter reaction flask. The air in the flask was removed completely by vacuumizing, keep introducing ultra pure CO₂ (99.999%) into the flask until the pressure in the flask approaches 0.1 - 0.3MPa, then close the flask. When the gas in the bottle was confirmed to be pure carbon dioxide analyzed by gas chromatography, switch on mini fountain pump, wherein the ionic liquid was scattered in the flask and thoroughly mixed with CO₂, and series chemical reactions take place. After continuous reaction for 3 hours, the gas in the flask was extracted for GC analysis. The chromatographic peaks were identified appearing near t=0.9 min and t=2.2 min respective, while the chromatographic peak near t=4.5 min (the characteristic peak for CO₂) disappeared completely, suggesting that CO₂ was converted to carbon monoxide and methane, wherein carbon monoxide was the major component which amounts to 70% of the products.

### Embodiment 4: Other Nitrogen-Containing Heterocyclic Compounds + Bimetallic Systems

Besides the above embodiment about the catalytic reduction of CO₂, Other similar imidazole + bimetallic systems also reduced carbon dioxide at room temperature to carbon monoxide, methane, or a combination of thereof, wherein the specific reactants, catalysts and productions are listed in table 3 (note: since 1-methyliminazole, 1-ethyliminazole, and ionic liquid of 1-butyl-3-methyl iminazolium bromide salt are in a liquid state themselves, these reaction system need no methanol, ethanol as solvent) .

Table 3

Imidazole with bimetallic and the catalytic reduction product of carbon dioxide and carbohydrates

| | | | | |
|---|---|---|---|---|
| Imidazole and its derivatives | Solvent | Metal 1 (M₁) | Metal 2 (M₂) | Product |
| Imidazole (8 g) | Ethanol (mL) | Sn powder (0.5 g) | Zn powder (2.5 g) | CO |
| 1-ethyliminazole(60ml) | | Cu powder (0.5 g) | Zn powder (2.5 g) | CO |
| 1-methyliminazole(60ml) | | Ag powder (0.5 g) | Fe powder (2.5 g) | CO+CH₄ |
| 4-methyliminazole(60ml) | ethanol (mL) | Cu powder (0.5 g) | Fe powder (2.5 g) | CO |
| 1-allyiminazole (60ml) | -- | Ni powder (0.5 g) | Al powder (2.5 g) | CO |
| 1-ethyliminazole (60ml) | -- | Pb powder (0.5 g) | Mn powder (2.5 g) | CH₄ |
| 1-methyliminazole(60ml) | -- | Cu powder (0.5 g) | Fe powder (2.5 g) | CO |
| 1-methyliminazole(60ml) | -- | Cu powder (0.5 g) | Al powder (2.5 g) | CO |
| 1-methyliminazole(60ml) | -- | Cu powder (0.5 g) | Mg powder (2.5 g) | CO |
| 1-methyliminazole(60ml) | -- | Ag powder (0.5 g) | Fe powder (2.5 g) | CO |
| 1-methyliminazole(60ml) | -- | Ag powder (0.5 g) | Zn powder (2.5 g) | CO |
| 1-methyliminazole(60ml) | -- | Ag powder (0.5 g) | Al powder (2.5 g) | CO |
| 2-methyliminazole(8 g) | methanol (mL) | Cu powder (0.5 g) | Al powder (2.5 g) | CH₄ |
| 1-butyl-3-methyl iminazolium bromide salt (60ml) | -- | Co powder (0.5 g) | Al powder (2.5 g) | CO+CH₄ |
| 1-ethyul-3-methyliminazolium tetrafluroborate salt(60ml) | -- | Ag powder (0.5 g) | Zn powder (2.5 g) | CO+CH₄ |
| 1,2,4-triazole (8 g) | ethanol (mL) | Sn powder (0.5 g) | Al powder (2.5 g) | CO |
| 1-benzyliminazole (8 g) | methanol (mL) | Ag powder (0.5 g) | Mn powder (2.5 g) | CH₄ |
| Histamine (8 g) | ethanol (mL) | Cu powder(0.5 g) | Fe powder (2.5 g) | CO+CH₄ |
| 1-benzyliminazole (8 g) | methanol (mL) | Ag powder (0.5 g) | Al powder (2.5 g) | CO |
| 1-butyliminazole (8 g) | ethanol (60ml) | Ag powder (0.5 g) | Mn powder (2.5 g) | CO |
| acetonitrile (1-iminazole group) (8 g) | methanol (mL) | Pb powder (0.5 g) | Zn powder (2.5 g) | CH₄ |
| 1,2-dimethylimidazole (8) | H₂O (mL) | Ni powder (0.5 g) | Fe powder (2.5 g) | CO+CH₄ |
| 1-acetylimidazole (8 g) | H₂O (mL) | Co powder (0.5 g) | Fe powder (2.5 g) | CO |

### Embodiment 5: Capturing of Carbon Dioxide and Its Conversion In-Situ

Flue gas emitted from on-site plants (power plants, aluminum plants, cement plants, etc.) was generally a mixture of carbon dioxide, oxygen and nitrogen, wherein the content of carbon dioxide was about 15%. Super pure carbon oxide was achieved through adsorption, desorption, and compression which involve high press condition and energy consuming process. If the carbon oxide capture and its reduction and conversion can be integrated, it will not only be desirable to reduce the equipment cost and energy consumption, but also accomplish the catalytic reduction and conversion of carbon dioxide economically at room temperature and atmosphere pressure. The present invention describes the imidazole+bimetallic system which demonstrate excellent selectivity and rapid adsorption of carbon oxide, wherein the integrated catalytic reduction system of 1-methyliminazole+Cu+Zn was applied in the capturing of carbon dioxide and its conversion in-situ. At room temperature, 60 milliliter of 1-methylimidazole, 2.5 gram of zinc powder and 0.5 gram of copper powder were put into a one liter reaction flask. The air in the flask was removed completely by vacuumizing, then simulated flue gas (15% of carbon oxide and 85% of oxygen) was introduced into the imidazole+bimetallic system by bubbling, chromatorgraphic analysis data indicated that 90% of carbon dioxide of the flue gas can be adsorbed by the imidazole+bimetallic system. When the introducing of flue gas was finished, the oxygen in the flask was removed by vacuuming to make sure that the converted product was free of oxygen. Then the switch of mini fountain pump was turned on, wherein the imidazole suspension with metallic power was scattered in the flask and thoroughly mixed with CO₂, and series chemical reactions took place. After continuous reaction for 2 hours, the gas in the flask was extracted for GC analysis, and the chromatographic analysis data demonstrate that 100% of carbon monoxide filled with the flask, which suggests carbon dioxide was converted with high efficiency and selectivity. This innovative process avoids the adsorption and desorption steps, and make sure that the economically integrated of adsorption and conversion of carbon oxide.

### Embodiment 6: Conversion of Carbohydrate to Carbon Monoxide

Besides the conversion of carbon oxide to carbon monoxide or methane, natural carbohydrates such as glucose and cellulose can be converted to desired fuel and economical chemical reagents.

At room temperature, 30 milliliter of 1-methylimidazole, 1 milliliter of H₂O, 2.5 gram of zinc powder,0.5 gram of copper powder, and 3 gram of glucose (or sucrose, starch, cellulose) were put into a one liter flask. The air in the flask was removed completely by vacuumizing, then the switch of magnetic stirrer was turned on, and series chemical reactions took place. After continuous reaction for 4 hours, the gas in the flask was extracted for GC analysis, and the chromatographic analysis data demonstrate that glucose, sucrose, starch and cellulose can be converted to carbon monoxide, wherein the conversion efficiency of glucose to carbon monoxide ranks first, with its maximum concentration of carbon monoxide approaching 10⁵ ppm.

The embodiment was an indirect utilization of carbon dioxide, wherein carbon dioxide was firstly converted to carbohydrate in chloroplast of green leaves through photosynthesis, then with the innovative catalytic reduction process, the synthesized carbohydrate was converted to energy materials or bio-diesel.

### Embodiment 7: Imidazole+Copper Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of copper powder were put into a one liter reaction flask. The air in the flask was removed completely by vacuumizing, then super pure carbon dioxide (99.999% of carbon oxide) was being introduced into the flask until the intensity of pressure approaches 0.1 - 0.3MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, 4 hours, and 8 hours respectively the gas in the flask was extracted for GC analysis, and the chromatographic peak still appeared around t = 4.5 min, which suggests no conversion of carbon dioxide in the flask.

### Embodiment 8: Imidazole+Nickel Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of nickel powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, 4 hours, and 8 hours respectively the gas in the flask was extracted for GC analysis, and the chromatographic peak still appeared around t = 4.5 min, which suggests no conversion of carbon dioxide in the flask.

### Embodiment 9: Imidazole+Silver Catalytic System

At room temperature, 60 milliliter of 1-ethylimidazole (or imidazole, 2-methylimidazole, 1-allyiminazole, 1-ethyul-3-methyliminazolium tetrafluroborate salt) and 1 gram of silver powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, 4 hours, and 8 hours respectively the gas in the flask was extracted for GC analysis, and the chromatographic peak still appeared around t = 4.5 min, which suggests no conversion of carbon dioxide in the flask.

### Embodiment 10: Imidazole+Cobalt Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of cobalt powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, 4 hours, and 8 hours respectively the gas in the flask was extracted for GC analysis, and the chromatographic peak still appeared around t = 4.5 min, which suggests no conversion of carbon dioxide in the flask.

### Embodiment 11: Imidazole+Tin Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of tin powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, 4 hours, and 8 hours respectively the gas in the flask was extracted for GC analysis, and the chromatographic peak still appeared around t = 4.5 min, which suggests no conversion of carbon dioxide in the flask.

### Embodiment 12: Imidazole+Zinc Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of zinc powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, the gas in the flask was extracted for GC analysis, and the chromatographic peaks appeared around t = 0.9 min and t = 4.5 min respectively, which suggests 7.5% of conversion rate of carbon dioxide to carbon monoxide in terms of the calculation the chromatographic peak area. The conversion rate of carbon dioxide to carbon monoxide approaches 35% when the system was continuously mixed for 24 hours in the flask.

### Embodiment 13: Imidazole+Aluminum Catalytic System

At room temperature, 60 milliliter of 1-ethylimidazole (or imidazole, 2-methylimidazole, 1-allyiminazole, 1-ethyul-3-methyliminazolium tetrafluroborate salt) and 2 gram of aluminum powder were put into a one liter reaction flask. The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, the gas in the flask was extracted for GC analysis, and the chromatographic peaks appeared around t = 2.2 min and t = 4.5 min respectively, which suggests 3.8% of conversion rate of carbon dioxide to methane in terms of the calculation the chromatographic peak area. The conversion rate of carbon dioxide to methane approaches 16.6% when the system was continuously mixed for 24 hours in the flask.

### Embodiment 14 a Series of Imidazole+Bimetallic Catalytic System (Illustrates as Table 4) With Different Metallic Proportion

At room temperature, 60 milliliter of 1-methylimidazole and certain grams of metal #1(M₁) and metal #2 (M₂) (with different proportions of the two metals, shown in Table 4) were put into a one liter reaction flask.

Table 4

the effect of metallic proportion on the conversion of carbon dioxide (reaction temperature: room temperature; reaction time: 2 hours)

| No. | M₁ | M₂ | M₁/M₂ | CO₂ conversion |
|---|---|---|---|---|
| 1 | Cu powder (10 g) | Fe powder (2.5 g) | 4:1 | 99.23 % |
| 2 | Cu powder (5 g) | Fe powder (2.5 g) | 2:1 | 99.98 % |
| 3 | Cu powder (2.5 g) | Fe powder (2.5 g) | 1:1 | 99.91 % |
| 4 | Cu powder (0.5 g) | Fe powder (2.5 g) | 1:5 | 99.92 % |
| 5 | Cu powder (0.25 g) | Fe powder (2.5 g) | 1:10 | 99.87 % |
| 6 | Cu powder (0.25 g) | Fe powder (5 g) | 1:20 | 99.65 % |
| 7 | Cu powder (0.25 g) | Fe powder (10 g) | 1:40 | 99.42 % |
| 8 | Cu powder (0.25 g) | Fe powder (20 g) | 1:80 | 99.33 % |
| 9 | Cu powder (0.25 g) | Fe powder (25 g) | 1:100 | 99.53 % |
| 10 | Cu powder (0.25 g) | Fe powder (50 g) | 1:200 | 99.68 % |
| 11 | Cu powder (10 g) | Zn powder (2.5 g) | 4:1 | 99.74 % |
| 12 | Cu powder (5 g) | Zn powder (2.5 g) | 2:1 | 99.13 % |
| 13 | Cu powder (2.5 g) | Zn powder (2.5 g) | 1:1 | 99.34 % |
| 14 | Cu powder (0.5 g) | Zn powder (2.5 g) | 1:5 | 99.26 % |
| 15 | Cu powder (0.25 g) | Zn powder (2.5 g) | 1:10 | 99.45 % |
| 16 | Cu powder (0.25 g) | Zn powder (5 g) | 1:20 | 99.88 % |
| 17 | Cu powder (0.25 g) | Zn powder (20 g) | 1:80 | 99.95 % |
| 18 | Cu powder (0.25 g) | Zn powder (25 g) | 1:100 | 99.36 % |
| 19 | Cu powder (0.25 g) | Zn powder (50 g) | 1:200 | 99.23 % |
| 20 | Ag powder (10 g) | Zn powder (2.5 g) | 4:1 | 99.17 % |
| 21 | Ag powder (2.5 g) | Zn powder (2.5 g) | 1:1 | 99.85 % |
| 22 | Ag powder (0.5 g) | Zn powder (2.5 g) | 1:5 | 99.39 % |
| 23 | Ag powder (0.25 g) | Zn powder (2.5 g) | 1:10 | 99.50 % |
| 24 | Ag powder (0.1 g) | Zn powder (25 g) | 1:250 | 99.76 % |

The air in the flask was removed completely by vacuumizing, then super pure carbon dioxide (99.999% of carbon oxide) was being introduced into the flask until the intensity of pressure approaches 0.1 - 0.3 MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 2 hours, the gas in every flask was extracted for GC analysis, and the chromatographic peaks appeared around t = 0.9 min and t = 4.5 min respectively, which illustrates n% of conversion rate of carbon dioxide to methane in terms of the calculation the chromatographic peak area, (as illustrated in table 4), which suggests the proportion of two metals applied in the above reaction performing no effect on the catalytic effect of the imidazole+bimetallic in the reduction of carbon dioxide.

### Embodiment 15: Series of Bimetallic+Imidazole Catalytic Systems With Different Concentration of Solid Imidazole

At room temperature, 60 milliliter of ethanol, n gram of 4-methylimidazole (the value of n was listed in table 5), 0.1 or 0.5 gram of metal #1(M₁) and 2.5 gram of metal #2 (M₂) were put into a one liter reaction flask. The catalytic systems comprise of 2.5 gram of M₁, 0.1 gram of M₂, and different gram of 4-methylimidazole which is illustrated in table 5.

Table 5

The effect of concentration of imidazole compound on the conversion rate of carbon oxide (reaction temperature, room temperature; reaction time, 2 hours)

| No. | imidazole | solvent | Metal #1 (M₁) | Metal #2 (M₂) | CO₂ conversation rate |
|---|---|---|---|---|---|
| 1 | 4-methylimidazole (5 g) | Ethanol(mL) | Ag powder (0.1 g) | Mg powder (2.5 g) | 26.45 % |
| 2 | 4-methylimidazole (10 g) | Ethanol(mL) | Ag powder (0.1 g) | Mg powder (2.5 g) | 27.00 % |
| 3 | 4-methylimidazole (20 g) | Ethanol(mL) | Ag powder (0.1 g) | Mg powder (2.5 g) | 26.32 % |
| 4 | 4-methylimidazole (4 g) | Ethanol(mL) | Ag powder (0.1 g) | Mg powder (2.5 g) | 25.97 % |
| 5 | 4-methylimidazole (5 g) | Ethanol(mL) | Co powder (0.5 g) | A1 powder (2.5 g) | 13.35 % |
| 6 | 4-methylimidazole (10 g) | Ethanol(mL) | Co powder (0.5 g) | A1 powder (2.5 g) | 14.54 % |
| 7 | 4-methylimidazole (20 g) | Ethanol(mL) | Co powder (0.5 g) | A1 powder (2.5 g) | 13.70 % |
| 8 | 4-methylimidazole (40 g) | Ethanol(mL) | Co powder (0.5 g) | A1 powder (2.5 g) | 13.20 % |

The air in the flask was removed completely by vacuumizing, then super pure carbon dioxide (99.999% of carbon oxide) was being introduced into the flask until the intensity of pressure approaches 0.1 - 0.3MPa, and the flask was sealed. The gas chromatographic instrument was applied to analyze the composition in the flask, the chromatographic peak appeared around t = 4.5 min, the gas in the flask was identified as carbon dioxide by comparison with the standard gas. The switch of mini fountain pump was turned on, wherein the imidazole suspension with mono-metallic power was scattered in the flask and thoroughly mixed with CO₂, and series chemical reactions took place. After continuous reaction for 3 hours, the gas in every flask was extracted for GC analysis, and the chromatographic peaks appeared around t = 2.2 min and t = 4.5 min respectively, which illustrates n% of conversion rate of carbon dioxide to carbon monoxide in terms of the calculation the chromatographic peak area, wherein n ranges from 13.20 to 27.00 (as illustrated in table 5), which suggests that the concentration of solid imidazole dissolved in ethanol plays no significant effect on the catalytic effect in the carbon oxide reduction.

### Embodiment 16: Reaction Mechanism

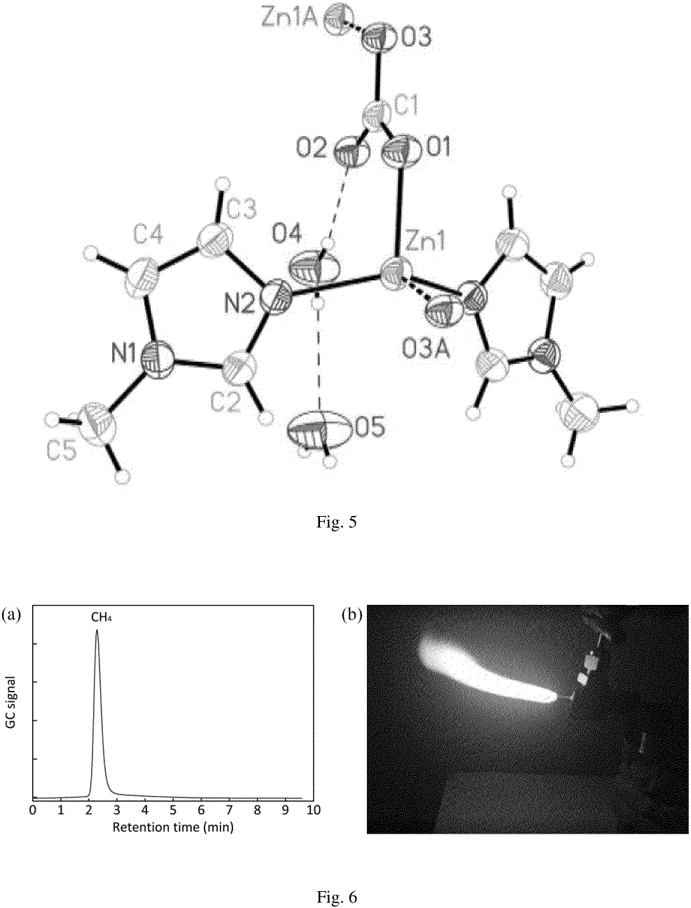

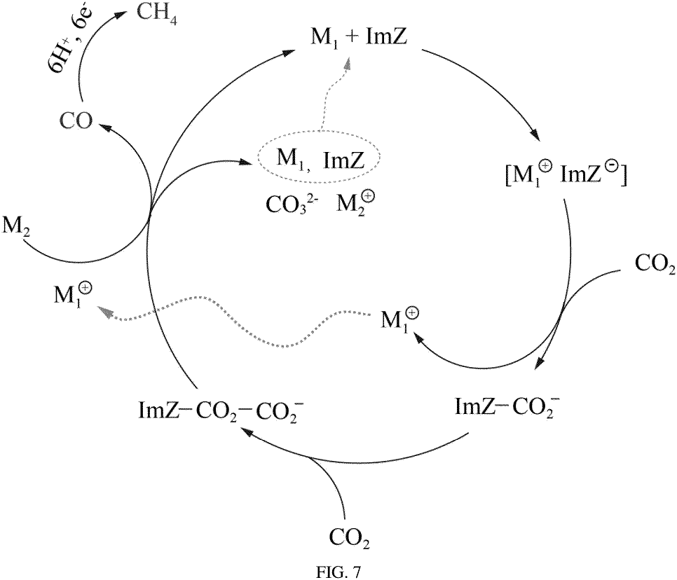

Taking imidazole compounds as an example, the reaction mechanism of the present invention is as follows: imidazoline compounds (imidazoline, abbreviated as ImZ) are compounds with aromatic structure characteristics and have the ability to accept electrons. When ImZ is in contact with some metal M₁, electron transfer can occur between them (FIG. 7), imidazole is an electron acceptor, and metal is an electron donor. After the charge transfer, a charge transfer complex is formed in the system, and imidazole becomes a negatively charged anion [ImZ] Θ with high activity, and its free radical signal is shown in FIG. 3a (detection results in Example 1). The carbon atoms in carbon dioxide have an electron-deficient structure, so [ImZ] Θ will activate carbon dioxide molecules to form carbon dioxide anion radicals (FIG. 3b, the detection results in Example 1). The free radical continues to get an electron from another more active metal M₂ in the system, and finally disproportionates into a carbon monoxide molecule and a carbonate ion. Carbonate and ImZ and metal M₂ generate M₂CO₃ (ImZ) x (x=1, 2, 3), for example, the reaction product in Example 1 is Zn (CO₃) (C₄N₂H₆)₂•2H₂O (FIG. 5). The generated carbon monoxide molecules may continue to undergo the (6H⁺, 6e⁻) process until methane molecules are generated. The catalytic principles of other nitrogen-containing heterocyclic compounds are similar.

The reaction equation is as follows: M₂+xImZ+2CO₂==CO+M₂CO₃(ImZ) x, (x=1,2,3)

In one or more embodiments of the present application, M₁ is first complexed with ImZ to form a free radical, has the ability to absorb and activate carbon dioxide, and reacts with M 2 and ImZ to form CO and M₂CO₃(ImZ) x. In this application, M₁ and xImZ have the effect of catalytic activation after complexation, and a small amount is sufficient. Therefore, M1 (metal 1) plays an auxiliary catalytic role, and is always recycled and will not be consumed. ImZ not only plays a catalytic role but also participates in the reaction, M₂ is a reactant, and finally combines with ImZ to generate carbonate.

1. A composition for catalytic reduction of carbon dioxide or carbohydrates comprising a nitrogen heterocyclic compound and at least two metal elements, wherein, a first metal element, performing as a reactant, has more reactivity than a second metal element which acts as co-catalyst.

2. The composition according to claim 1, wherein the nitrogen heterocyclic compound is at least one of the following: imidazole, 1-methylimizazole, 1-ethylimizazole, 1-ethyul-3-methylimidazolium tetrafluroborate salt, 4-methylimidazole, 1-allyimidazole, 2-methylimidazole, 1-butyl-3-methyl imidazolium bromide salt, 1-benzylimidazole, histamine, 1-butylimidazole, acetonitrile(1-imidazole group), 1,2-dimethylimidazole, 1-acetylimidazole, and 1,2,4-triazole.

3. The composition according to claim 1, wherein the two metal elements are different.

4. The composition according to claim 2, wherein, the second metal element, acting as the co-catalyst, is one of the following metal elements: tin, copper, silver, nickel, cadmium, cobalt and lead.

5. The composition according to claim 2, wherein, the first metal element, acting as the reactant, is one of the following metal elements: zinc, iron, aluminum, manganese, magnesium, nickel, tin.

6. A method of using the composition metal according to claim 1 for catalytic reduction of carbon dioxide or carbohydrates.

7. A method of using the composition metal comprising the step of: mixing a substrate with the composition of claim 1, reacting to convert CO₂ and carbohydrate to CO and methane.

8. The method according to claim 7, wherein, the nitrogen heterocyclic compound is solid under room temperature, dissolving the nitrogen heterocyclic compound in a solvent.

9. The method according to claim 8, wherein the solvent is water, methanol, or ethanol.

10. The method according to claim 7, wherein, CO₂ is pure CO₂ or waste gas containing carbon dioxide.
